(19) United States
(12) Patent Application Publication
(10) Pub. No.: US 2004/0081617 A1
(43) Pub. Date: Apr. 29, 2004

(54) Radio-labelled ferrite particles and methods for the manufacture and use thereof

(76) Inventors: Rodney James Browitt, New South Wales (AU); Timothy John Senden, New South Wales (AU)

Correspondence Address: BIRCH STEWART KOLASCH & BIRCH, PO BOX 747, FALLS CHURCH, VA, 22040-0747, US

(21) Appl. No.: 10/415,490

(22) PCT Filed: Oct. 24, 2001
(86) PCT No.: PCT/AU01/01365

(30) Foreign Application Priority Data
Oct. 30, 2000 (AU) PR 1131

(51) Int. Cl. A61K 51/00

(52) U.S. Cl. 424/1.11

(57) ABSTRACT

A method for the production of radio-labelled ferrite nanoparticles for use in medical imaging and radiotherapy comprising the steps of: a) adding an aqueous solution containing Fe²⁺ and Fe³⁺ ions and at least one radioisotope to an alkaline solution and agitating the mixture to form a precipitate comprising ferrite particles labelled with the at least one radioisotope; and b) isolating and washing the precipitated labelled particles, wherein said radioisotope is a radioisotope which functions as a radiotracer isotope and a radiotherapy isotope or said radioisotope includes at least one radiotracer isotope and at least one radiotherapy isotope.

The present invention relates to radio-labelled ferrite particles and methods of manufacturing same. The invention further relates to uses of such particles for medical imaging and therapy.

Ferrimagnetic nanoparticles are known. Such particles have been used previously in hyperthermia therapy for human cancers. The principle used in this case involves the induction of intracellular hyperthermia by external application of an oscillating electromagnetic field after endocytosis of magnetic nanoparticles by tumour cells. This method of treatment has particularly been pursued for treatment of malignant brain tumours and oral cancers.

It would be desirable in such applications to quantify localisation of nanoparticles in the target tissues. As such, the inventors have now provided a method of radiolabelling such ferrimagnetic nonoparticles. It has also been found that the labelled nanoparticles may also have a wider usefulness in other applications, thus permitting better imaging of tumours based on the selective rapid uptake of the particles by tumour cells with gamma camera imaging or scintigraphy; localised radiotherapy of tumours using high density labelling of the nanoparticles; and radio-guided surgery for more effective resection of poorly defined tumours.

According to one aspect of the invention there is provided a method for the production of radio-labelled ferrite nanoparticles comprising the steps of:

a) adding an aqueous solution containing Fe²⁺ and Fe³⁺ ions and a radioisotope to an alkaline solution and agitating the mixture to form a precipitate comprising ferrite particles labelled with the radioisotope; and

b) isolating and washing the precipitated labelled particles.

There is also provided a radio-labelled ferrite particle produced by the method of the immediately preceding paragraph.

The aqueous solution, including saline solutions, which has preferably been degassed to a greater extent of oxygen, may contain Fe²⁺ and Fe³⁺ ions, preferably as FeCl₂ and FeCl₃, in a molar ratio of about 1:2, at a concentration of around 1M or less in Fe²⁺. The radioisotope may be a radiotracer isotope or a radiotherapy isotope preferably at a concentration lower than the concentration of Fe²⁺. For example, the radioisotope may include an imaging radiotracer isotope selected from the group consisting of ^{99m}Tc, ¹¹¹In, ⁶⁷Ga and ²⁰¹Tl, or may include a radiotherapy isotope selected from the group consisting of ¹⁸⁸Re, ⁶⁴Cu, ¹⁹⁸Au, ⁹⁰Y and ¹⁶⁶Ho. In a particularly preferred embodiment, the radioisotope is ^{99m}Tc as pertechnetate anion. It will be understood, however, that the invention is not limited to the above list and that other isotopes may be used. Further, it has been found that ferrite will take up almost any element, including anions (e.g. TcO₄⁻). Such elements are considered to fall within the ambit of the present invention.

The alkaline solution to which the aqueous solution is added in step a) is preferably 1M sodium hydroxide solution. Agitation of the solution formed results in the precipitation of a dark precipitate comprised of magnetite. Development of the precipitate may be assisted by heating the solution to a temperature of around 70° C.

The product may be purified by separation in a magnetic field, by centrifugation or by filtration and can be washed at this stage, or re-dispersed and concentrated for further washing. This is done to remove non-incorporated reagents and radio-isotopes, and to change the type of medium the product is to be dispersed into. Re-dispersion can be affected by mechanical or ultrasonic agitation. The deposition from solution of, or reaction with an amphiphile, organic or inorganic polymer, or colloid can enhance stabilisation and affect biological binding affinity. The nature of the amphiphile, organic or inorganic polymer, or colloid can be selected to increase specificity of binding to a region or protein.

In a preferred embodiment, the isolation and washing step b) is carried out initially through with an amphiphile, organic or inorganic polymer, or colloid can enhance stabilisation and affect biological binding affinity. The nature of the amphiphile, organic or inorganic polymer, or colloid can be selected to increase specificity of binding to a region or protein.

In a preferred embodiment, the isolation and washing step b) is carried out initially through the application of an external magnetic field, the precipitate being washed while trapped in the magnetic field. After washing, the precipitate is then redispersed in a medium, such as an isotonic saline or glucose solution, using ultrasonics. The precipitate may then be autoclaved if sterilisation is required.

Magnetic separation of the product is achieved by the placement of a magnetic field, for example by a permanent rare earth type magnet, on the exterior of the vessel trapping the precipitate against the vessel wall.

According to another aspect of the invention there is provided radio-labelled ferrimagnetic nanoparticles for use in medical imaging and therapy comprising magnetite and a radioisotope, the radioisotope being entrapped in the magnetite, preferably through precipitation of a solution comprising Fe²⁺ and Fe³⁺ ions and the radioisotope.

The radioisotope may be selected from the imaging radiotracer isotopes and radiotherapy isotopes described above. The particle size of the ferromagnetic nanoparticles may be any suitable size which facilitates their use for the desired applications, that is for medical imaging and therapy. In a preferred embodiment, the average particle size is from 5 to 200 nanometres. Generally, the average particle size will be less than 50 nanometres.

Advantageously the particles retain better than 99% of their entrained activity (pertechnetate) in the pH range 1-14, in boiling NaOH at pH>14, after 15 minutes exposure to ultrasonics, or after autoclaving. In this regard, the level of “free” or evolved pertechnetate can be determined by radiometric chromatography.

The invention in another aspect provides the use of radio-labelled ferrite nanoparticles prepared by the method of the invention or as described above in medical imaging and/or therapy.

The product may be sterilised via autoclaving or filtration. It may be injected, inhaled as a fine dispersion, or ingested. The total administered radioactivity is a measure of the dose of the product, and the distribution of the product can be determined by radiation monitor, scintigraphy, including emission tomography, or magnetic imaging such as MRI.

The total dose of product and the specific activity can be varied depending on application. For hyperthermia the particle dose may be high, less than about 1 g, but the radio-activity, in the form of a radio-tracer may be as low as the detectable level. For radiotherapy, the particle dose may be low, <1 μg, but the radioactivity can be at a therapeutic level, and may also include a detectable level of a suitable radio-tracer. Administration of the product via injection or otherwise into a region to undergo radiological or radiofrequency therapy, is followed by determination of the distribution of the product around the site of interest by mapping the radio-activity from the included radio-isotope.

Radiometric assaying of magnetically separated product demonstrates that >99% of the initial radioactivity, from pertechnetate, is stable entrained within the product. Similarly with thin layer chromatography, using either water or methylethylketone as the carrier, >99% of the activity is immobilised at the point of origin.

In order to further describe embodiments of the present invention, reference will now be made to the accompanying drawings in which:

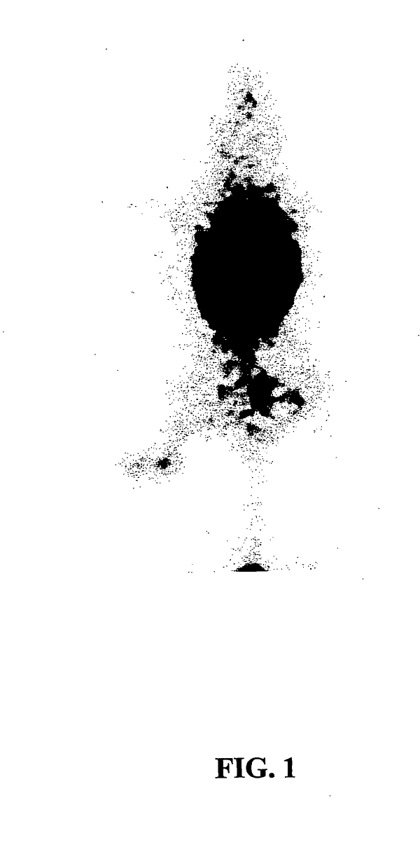

FIG. 1 illustrates a rat tail vein injection showing whole body scintigraphy collected on a gamma camera or tumour is located in the left leg of the rat;

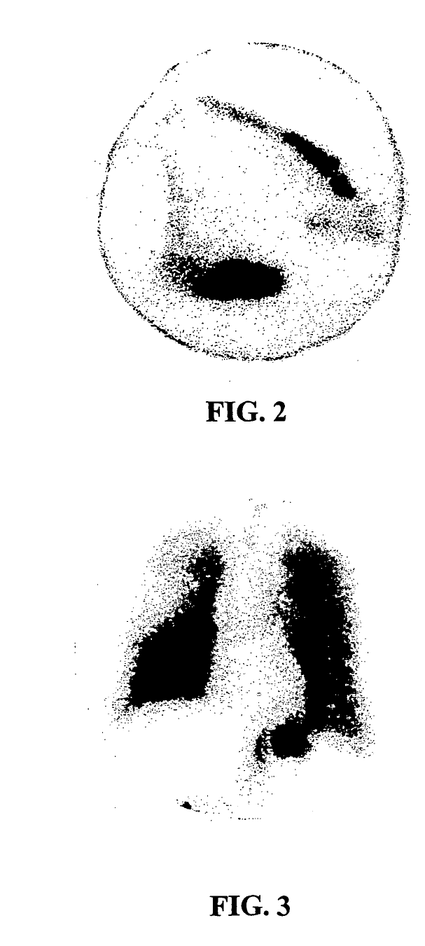

FIG. 2 illustrates human lungs ventilated with a wet aerosol made by ultrasonic dispersion of a saline suspension of the nanoparticles of the invention and imaged with a gamma camera;

FIG. 3 illustrates a human bowl imaged by ingesting a saline suspension of the nanoparticles of the invention and imaged with a gamma camera;

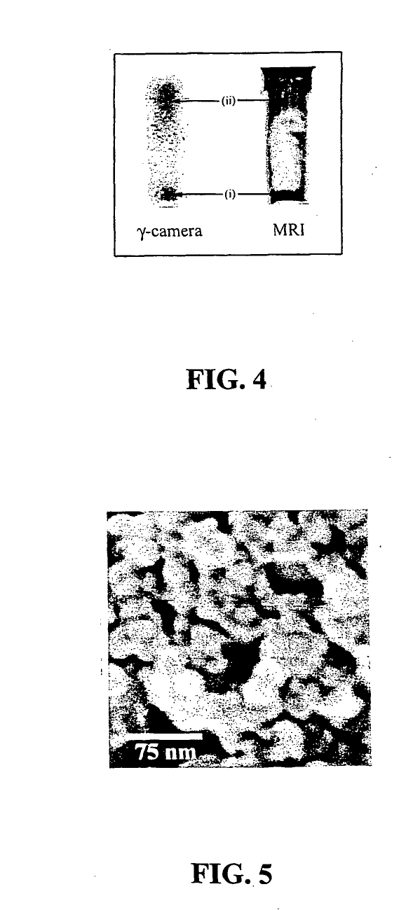

FIG. 4 illustrates a scintigraphic-MRI phantom; and

FIG. 5 illustrates a scanning electron micrograph of the nanoparticles of the invention.

Referring to FIGS. 1-3, it will be seen that good imaging of the radio-labelled ferrite particles may be achieved using a gamma camera. These figures also illustrate the effectiveness of the particles when administered by injection, ventilation and ingestion.

Referring to FIG. 4, the left image is a scintigraphic image of a gelatin phantom containing ferrite particles entraining ^{99m}Tc in a striated pattern. The right is the same gel imaged with MRI. Region (i) shows a concentrated layer of the product at the bottom of the sample vial. Region (ii) shows a diffuse region of the product. The region in between (i) and (ii) shows slight intermixing. Total loading of product in the vial is around 500 μg per mL.

Referring briefly to FIG. 5, the scanning electron micrograph of the particles of the invention illustrates that the primary particles are of a particle size of about 30 nm.

### EXAMPLE

Several mLs of an aqueous solution, purged of O₂, containing 0.02 M FeCl₂ and a 0.01 M FeCl₃, the isotope to be encapsulated (e.g. 20 MBq of Na^{99m}TcO₄ in saline) and acidified with HCl to pH 4 or lower is made by dilution from stock reagents. This solution may then be added drop wise to a similar volume of a stirred 1 M NaOH solution heated at 70° C. The solution will darken immediately and should be maintained at this temperature for a few minutes. If the reaction is conducted at room temperature stirring should be maintained for not less than 5 min.

The product formed can be magnetically separated by placing a strong rare earth magnet on the exterior of the reaction vessel while the reagents and solution are decanted. Removing the magnet, re-dispersing the product in a liquid medium such as saline, and repeating the decanting step several times will remove residual reagents. Alternatively, filtration or centrifugation can also be used.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

The reference to any prior art in this specification is not, and should not be taken as, an acknowledgment or any form of suggestion that that prior art forms part of the common general knowledge in Australia.

Those skilled in the art will appreciate that the invention described herein is susceptible to variations and modifications other than those specifically described. It is to be understood that the invention includes all such variations and modifications which fall within its spirit and scope. The invention also includes all the steps, features, compositions and compounds referred to or indicated in this specification, individually or collectively, and any and all combinations of any two or more of said steps or features.

1. A method for the production of radio-labelled ferrite nanoparticles comprising the steps of:
a) adding an aqueous solution containing Fe²⁺ and Fe³⁺ ions and a radioisotope to an alkaline solution and agitating the mixture to form a precipitate comprising ferrite particles labelled with the radioisotope; and
b) isolating and washing the precipitated labelled particles.

2. A method according to claim 1, wherein the aqueous solution contains Fe²⁺ and Fe³⁺ ions, preferably as FeCl₂ and FeCl₃, in a molar ratio of about 1:2, at a concentration of around 1M or less in Fe²⁺.

3. A method according to claim 1, wherein the radioisotope is a radiotracer isotope or a radiotherapy isotope, and is preferably present at a concentration lower than the concentration of Fe²⁺.

4. A method according to claim 1, wherein the radioisotope includes an imaging radiotracer isotope selected from the group consisting of ^{99m}Tc, ¹¹¹In, ⁶⁷Ga and ²⁰¹Tl, or a radiotherapy isotope selected from the group consisting of ¹⁸⁸Re, ⁶⁴Cu, ¹⁹⁸Au, ⁹⁰Y and ¹⁶⁶Ho.

5. A method according to claim 4, wherein the radioisotope is ^{99m}Tc as pertechnetate anion.

6. A method according to claim 1, wherein the alkaline solution to which the aqueous solution is added in step a) is 1M sodium hydroxide solution.

7. A method according to claim 1, wherein formation of the precipitate is assisted by heating the solution to a temperature of about 70° C.

8. A method according to claim 1, wherein the isolation and washing step b) is carried out initially through the application of an external magnetic field, the precipitate being washed while trapped in the magnetic field, followed by redispersion of the precipitate in a medium, such as an isotonic saline or glucose solution, using ultrasonics.

9. A radio-labelled ferrite particle produced by the method of any one of the preceding claims.

10. Radio-labelled ferrimagnetic nanoparticles for use in medical imaging and therapy comprising magnetite and a radioisotope, the radioisotope being entrapped in the magnetite.

11. Radio-labelled ferrimagnetic nanoparticles according to claim 10, said particles being prepared through precipitation of a solution comprising Fe²⁺ and Fe³⁺ ions and the radioisotope.

12. Radio-labelled ferrimagnetic nanoparticles according to claim 10, wherein the average particle size of the nanoparticles is from 5 to 200 nanometres.

13. Radio-labelled ferrimagnetic nanoparticles according to claim 12, wherein the average particle size is less than 50 nanometres.

14. Radio-labelled ferrimagnetic nanoparticles according to claim 10, wherein the particles retain better than 99% of their entrained activity in the pH range 1-14, in boiling NaOH at pH>14, after 15 minutes exposure to ultrasonics, or after autoclaving.

15. Use of radio-labelled ferrite nanoparticles as defined in any one of claims 9 to 14 or prepared by the method of any one of claims 1 to 8 in medical imaging and/or therapy.

15. Radio-labelled ferrimagnetic nanoparticles according to claim 10, wherein the at least one radio-isotope is 64Cu alone.

16. Radio-labelled ferrimagnetic nanoparticles according to claim 10, wherein the at least one radio-isotope includes a radiotracer isotope and a radiotherapy isotope.

17. Radio-labelled ferrimagnetic nanoparticles according to claim 16, wherein the radiotracer isotope is selected from the group consisting of ^{99m}Tc, ¹¹¹In, ⁶⁷Ga and ²⁰¹Tl and the radiotherapy isotope is selected from the group consisting of ¹⁸⁸Re, ⁶⁴cu, 198Au, ⁹⁰Y and ¹⁶⁶Ho.

18. Use of radio-labelled ferrite nanoparticles as defined in any one of claims 9 to 14 or prepared by the method of any one of claims 1 to 8 in medical imaging and radiotherapy.
